(19) United States
(12) Plant Patent Application Publication
(10) Pub. No.: US 2024/0389490 P1
(43) Pub. Date: Nov. 21, 2024

(54) YUCCA PLANT NAMED 'MELLOW YELLOW'

(50) ;

(71) Applicant: Hans A. Hansen, Zeeland, MI (US)

(72) Inventor: Hans A. Hansen, Zeeland, MI (US)

(21) Appl. No.: 18/831,049

(22) Filed: May 17, 2024

(30) Foreign Application Priority Data
May 17, 2023 (EU) 20231079

(51) Int. Cl. A01H 6/00

(52) U.S. Cl. CPC A01H 6/00
USPC PLT/373

(57) ABSTRACT

A new and unique Yucca plant named ‘Mellow Yellow’ is characterized by a gracefully arching rounded mound of linear leaves that are flat with a broad, chartreuse-green, central band and margins of creamy-yellow to yellowish. The numerous leaves have micro marginal teeth and terminal mucro. The new plant is suitable for the garden or as a potted plant in the garden or home.

Botanical classification: Yucca gloriosa var. tristis Carrière.

Variety denomination: ‘Mellow Yellow’.

### CROSS-REFERENCE TO RELATED APPLICATION

This patent application claims the benefit of priority under 35 U.S.C § 119 of European Union (EU) Community Plant Variety Rights application Ser. No. 20/231,079 as filed on May 17, 2023, the contents of which are hereby incorporated by reference herein.

### STATEMENT REGARDING PRIOR DISCLOSURES UNDER 37 CFR 1.77 (b) (6)

The first public disclosures of the claimed plant, in the form of private sales, were made by Fitzgerald Nurseries to Globeplanter in France on Aug. 1, 2022. Plants for this sale were obtained from the inventor.

Prior to this, the new plant was listed on a website hosted by Plant Delights Nursery, Inc. on Dec. 11, 2020. The plant used for non-enabling picture was provided by the inventor and no plants were sold nor offered for sale. No plants of ‘Mellow Yellow’ have been sold or offered for sale, in this country or anywhere in the world nor has any disclosure of the new plant been made more than one year prior to the effective filing date of this application, and such sale or disclosure within one year was obtained either derived directly or indirectly from the inventor.

### BACKGROUND OF THE INVENTION

The present invention relates to the new and distinct Yucca plant, Yucca ‘Mellow Yellow’ that was discovered by the inventor at a wholesale perennial nursery in Waseca, Minnesota, USA as a whole plant mutation of a marginally variegated form of Yucca glorisosavar. tristis (not patented) marketed as Yucca recurvifolia ‘Variegata’ in 2004. The new plant has been successfully asexually propagated by division of the rhizomes and also by sterile shoot-tip tissue culture at a wholesale perennial nursery in Zeeland, Michigan. Both methods of asexual propagation have been found to produce stable and identical plants that maintain all the unique characteristics of the original plant in successive generations.

### BRIEF SUMMARY OF THE INVENTION

‘Mellow Yellow’ differs from all other Yucca known to the applicant. The nearest comparison plants are: ‘Walbristar’ U.S. Plant Pat. No. 17,653, ‘Tuszyuc001’ U.S. Plant Pat. No. 29,025, Y. gloriosa ‘Variegated’ (not patented), ‘Hinvargas’ U.S. Plant Pat. No. 12,192, ‘Gold Ribbons’ (not patented), and Yucca filamentosa ‘Bright Edge’ (not patented).

‘Walbristar’ has a shorter habit and slower growth rate. ‘Tuszyuc001’ has foliage with a more upright habit, more twisted, the center is more green and the edge more yellowish with some occasional reddish purple blush. ‘Variegated’ has more upright foliage with a deeper bluish-green center and a contrasting margin that begins yellowish and matures to a creamy edge. ‘Hinvargas’ which is frequently sold under the tradename MARGARITAVILLE™, has a reverse variegation pattern with a darker green leaf margin and yellow to yellow-green longitudinal bands in the center. ‘Gold Ribbons’ has darker green leaf margins and lighter yellowish leaf centers. ‘Bright Edge’ has foliage with darker centers and lighter margins, and the foliage is stiffer and less arching.

The parent plant has very narrow subtle chartreuse margin and the center is bluish-green. ‘Mellow Yellow’ is unique from all the above cultivars in possessing the following combined traits:
1. Numerous linear, flat, arching leaves;
2. Leaves have a broad, chartreuse-green, central band and margins of creamy-yellow to yellowish;
3. Moderate growth rate.

### BRIEF DESCRIPTION OF THE DRAWING

The photographs FIGS. 1, 2, and 3 of Yucca ‘Mellow Yellow’ demonstrate the overall appearance of the new plant including the unique traits as a four-year-old plant grown in a greenhouse in Zeeland, Michigan and FIG. 4 shows a twelve-year-old plant in a full-sun garden in Raleigh, North Carolina. The colors are as accurate as reasonably possible with color reproductions. Ambient light spectrum, temperature, source, and direction may cause the appearance of minor variations in color.

FIG. 1 shows the habit of the new plant in full flower from the side angle.

FIG. 2 shows a close-up of the foliage and scape base.

FIG. 3 shows a close-up of the upper scape with flowers and buds.

FIG. 4 shows the new plant in a full sun garden.

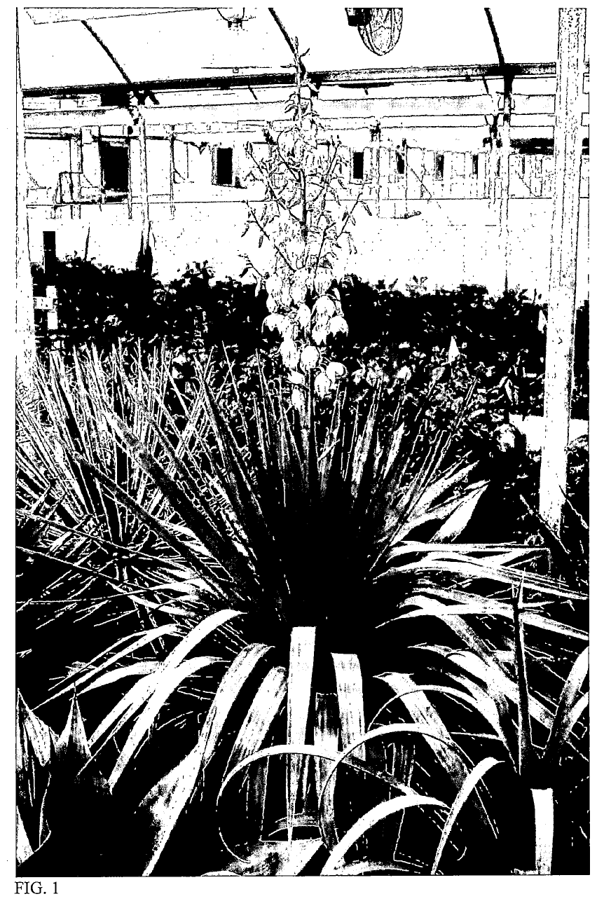

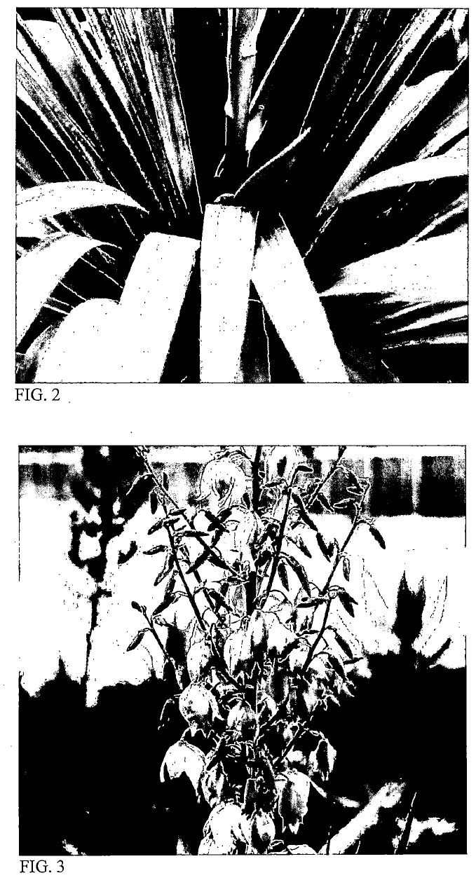

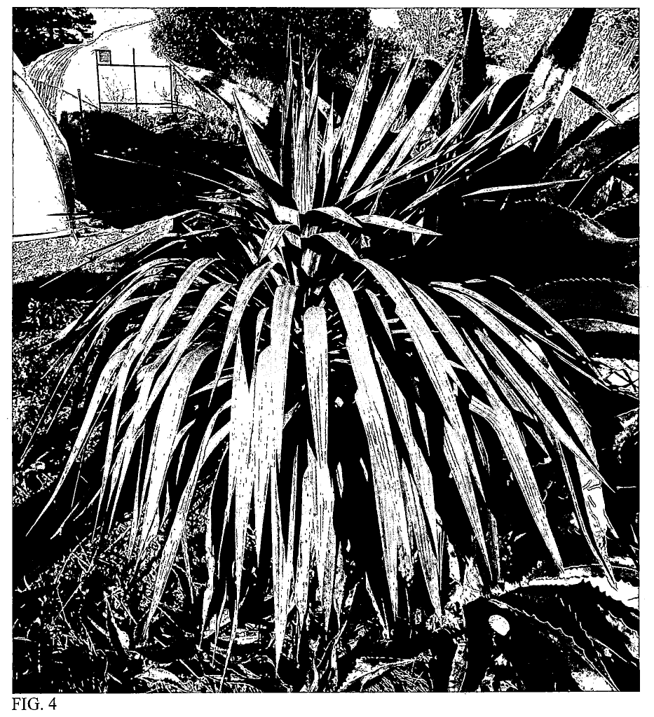

### DETAILED BOTANICAL DESCRIPTION

The following descriptions and color references are based on the 2015 edition of The Royal Horticultural Society Colour Chart except where common dictionary terms are used. The new plant, Yucca ‘Mellow Yellow’, has not been observed under all possible environments. The phenotype may vary slightly with different environmental conditions, such as temperature, light, fertility, moisture, and maturity levels, but without any change in the genotype. The following observations and size descriptions are of a four-year-old plant in a commercial wholesale greenhouse in Zeeland, Michigan and a twelve-year-old plant in a full-sun garden in Raleigh, North Carolina with supplemental water and fertilizer as needed.
Parentage: A whole plant mutation of a proprietary unnamed selection of Yucca gloriosa var. tristis;
Propagation: By shoot tip cuttings and sterile shoot-tip tissue culture;
Time to initiate roots from tissue culture: About 21 days;
Growth rate: Moderate;
Crop time: About 20 to 28 weeks to finish in a 3.8-liter container from a 35 mm tissue culture growing at about 21° C.;
Rooting habit: Fleshy, lightly branching, with roots up to 30 cm long;
Root color: Nearest RHS 158B;
Plant shape and habit: Succulent herbaceous perennial with basal rosettes of about 116 leaves radially emerging mostly upright from a central stem and arching distally, producing a radially-symmetrical rounded mound;
Plant size: Foliage height about 92 cm tall from the soil line to the top of the leaves, scape height to about 155 cm tall, and about 128 cm wide at the widest point just above soil level;
Stem: To about 3.5 cm across; covered with foliage;
Foliage description: Linear to lanceolate; simple; sessile; bi-laterally symmetrical; coriaceous; apex narrowly acute with terminal mucro; base truncate; dentate; spirally arranged; adaxial glabrous, abaxial scaberulous;
Mucro (apical spine): About 4.5 mm long and 0.7 mm wide; sharp; semi-flexible;
Mucro color: Between RHS 166A and RHS 177A;
Marginal teeth: Micro-dentate; protruding about 0.1 mm long from the margin and about 0.2 mm wide at the base, with average spacing about 1 mm apart; color too small to measure;
Leaf size: To about 73 cm long, about 42 mm wide toward middle, base about 16 mm wide; average about 62 cm long and 36 mm wide at center;
Foliage fragrance: None observed;
Leaf number: About 116 per plant;
Leaf blade color:
   Adaxial.—(Greenhouse) young margins between RHS 146D and RHS 151A, and mature margins between RHS 160A and RHS 153D; young centers nearest RHS N138C distally and between RS N138C and RHS 146C proximally, mature centers between RHS 146C and RHS 147C.
   Abaxial.—Young margins between RHS 146D and RHS N144A, and mature margins between RHS 160A and RHS 153D; young centers nearest RHS N138C distally and between RHS N138C and RHS 146C proximally.
   Adaxial (high light).—Young margins between 10A and RHS 11A, and young centers nearest RHS 146D; mature margins between RHS 11A and RHS 7B, and mature centers between RHS 152D and RHS 153D.
   Abaxial (high light).—Young margins between 10A and RHS 11A, young centers nearest RHS 146D; mature margins between RHS 11A and RHS 153D, and mature centers between RHS 146D and RHS N144A distally and between RHS 146D and RHS N144D proximally.
Petiole: Leaves sessile;
Veins: Parallel; color not distinct abaxial or adaxial;
Peduncle: Terete; glaucous; glabrous; stiff; strong; moderately branched; about 155 cm long and 16 mm diameter below first flowers;
Peduncle color: Basal portion before flowers nearest a blend between RHS 189B and RHS N138D, flowering portion nearest a blend between RHS 146D and RHS 145A, branches nearest RHS 146D;
Inflorescence: Flowering portion about 65 cm tall and about 21 cm across; with about 27 branches, branches to about 21 cm long and about 4 mm diameter at base decreasing distally; attitude main peduncle erect and branches outwardly at about a 45-degree angle above horizontal; with about 150 flowers per inflorescence;
Pedicel: Terete; glaucous; glabrous; arcuate downward; average about 25 mm long and 2 mm diameter;
Pedicel color: Between RHS 146D and RHS 145B;
Inflorescence bracts: About 27 per scape; lanceolate; simple, fleshy, glabrous; micro-dentate with flexible teeth; apex narrowly acute with semi-flexible mucro; base truncate, sessile, partially-clasping; to about 21 cm long and about 23 mm wide at base, decreasing distally; color identical to foliage;
Buds one day prior to opening: Oblong ellipsoidal; acute apex; truncate base; about 42 mm long and about 13 mm across at widest portion near middle;
Bud color: Nearest RHS 1D with apex midrib nearest RHS 146D with a slight blush of nearest RHS 187B; dorsal tepal midrib nearest RHS 145C;
Flower description: Perfect; incomplete; zygomorphic; about 48 mm long and opening to about 40 mm across; lasting about 3 to 4 days per flower; flowering period about three weeks in late summer or fall;
Inflorescence: panicle; Approximately 150 flowers per scape; with about 27 branches; branches to about 21 cm long and 4 mm diameter at base; attitude mostly upright;
Flower fragrance: Not detected;
Tepals: Six in two sets of three; oblong-lanceolate; with acute apex; truncate base; margin entire; glaucous and glabrous abaxial and adaxial; slightly concavo-convex; outer set to about 46 mm long and 19 mm wide near middle, and 6 mm wide at base; inner set about 50 mm long, 20 mm wide near middle, and 6 mm wide at base;
Tepal color: Outer set adaxial center midrib nearest RHS 4D when first open and maturing to between RHS 138B and RHS NN155B, outer portion nearest RHS NN155A; outer set abaxial midrib nearest RHS N144D distally and nearest RHS 146D proximally, blushed at apex with nearest RHS 187B; inner set adaxial proximally nearest RHS 145D and distally nearest RHS 4D in the center with outer portion nearest RHS NN155B; inner set abaxial proximally near center nearest RHS N144D and toward margins nearest RHS NN155B, distally near center nearest RHS 4D and near margin nearest RHS NN155B;
Androecium: Six;
   Filaments.—Six; applanate; straight; micro-glandular in proximal 17 mm; about 21 mm long, 3 mm wide and 1.8 mm thick near apex, and 2 mm wide and 1.6 mm thick at base; color nearest RHS NN155A.
   Anther.—Basifixed; longitudinal; globose; about 1 mm diameter when open, prior to dehiscing about 3 mm long and 2 mm across; color nearest RHS NN155A.
   Pollen.—Abundant; color nearest RHS 11D.
Gynoecium: Single; three-chambered; superior; to about 28 mm long;
   Style.—Three; 6 mm long and 3 mm wide at base; color between RHS 1D and RHS 160D.
   Stigma.—Bifid; about 2 mm wide, 1.5 mm long, and 1 mm thick; color nearest RHS NN155A.
   Ovary.—Superior.
Fruit and seed: Not yet observed;
Disease resistance and pest: Yucca ‘Mellow Yellow’ has not been observed to be resistant or susceptible to diseases and pests beyond that which is normal for other Yucca. The new plant is xeromorphic and survives well with minimal water once established. The new plant is tested to be winter hardy to USDA zone 7. Full extent of winter hardiness has not been tested.

It is claimed:

1. A new and distinct cultivar of ornamental Yucca plant named ‘Mellow Yellow’ as herein described and illustrated.
